(19) United States
(12) Patent Application Publication
(10) Pub. No.: US 2008/0025953 A1
(43) Pub. Date: Jan. 31, 2008

(54) Vigor Enhancement of Animals Via Administration of Stem Cells

(76) Inventors: Kiminobu Sugaya, Winter Park, FL (US); Angel Alvarez, Orlando, FL (US)

Correspondence Address: Beusse Wolter Sanks Mora & Maire, 390 N. ORANGE AVENUE, SUITE 2500, ORLANDO, FL, 32801, US

(21) Appl. No.: 11/459,760

(22) Filed: Jul. 25, 2006

(51) Int. Cl. A61K 35/30

(52) U.S. Cl. 424/93.7

(57) ABSTRACT

Disclosed herein are methods for increasing the vigor of nonhuman vertebrates utilizing stem cells. Unlike methods designed to treat disease conditions, the methods exemplified herein teach the utilization of stem cells for increasing vigor in animals not necessarily for the purpose of treating a disease, but can include application to clinically healthy animals.

### BACKGROUND

Much research is being conducted to study stem cells and to devise ways of utilizing stem cells in treating various neurological pathologies and injuries, as well as pathologies of other organ systems. It is generally recognized that stem cell technologies hold tremendous promise for ultimately treating and even curing neurologically related diseases, injuries or dysfunctions. It is understandable why stem cell research has focused on these areas. However, finding new and more diverse ways of utilizing stem cells is an ongoing challenge. As an example of a possible, unique application of stem cell research, it has recently been suggested that stem cells could be used in the bioengineering of cosmetic surgery related implants, including breast implants (http://www.newscientist.com/article.ns?id=dn7028). It is important that stem cell research be directed to beneficial areas that do not necessarily include the traditional areas of developing treatments and cures for disease and injuries.

### DETAILED DESCRIPTION

The subject invention is based on the inventors' realization that relatively little research and development has been conducted in the area of stem cell-based applications for healthy individuals or subjects. Furthermore, relatively little research and development has been conducted toward devising veterinarian-related uses of stem cells. The small animal veterinarian market is significant. The attachment that people develop toward their pets can be extremely powerful, and in many cases the love they feel is as strong as it would be for a child or other family member. As pets age, it is natural that they become less active and energetic. This is due in part to many animals not receiving sufficient exercise over the course of their lifetime, and due in part to the natural aging process. As pets get older they have a propensity to become obese, which is somewhat related to an animal's lower energy levels. Providing a pet the opportunity to experience higher energy levels even in the later years of a pet's life would be a cherished experience for many pet owners.

Accordingly, in one embodiment, the subject invention pertains to a method of increasing vigor of an animal that comprises the administration of a vigor-enhancing composition that contains autologous or exogenous, stem cells, or both and a pharmaceutically acceptable carrier. The method embodiment is not specifically directed to treating a pathological condition, injury or disease state but rather the rejuvenation of an animal. While much stem cell basic research is conducted in laboratory animals, mainly rodents, such research is designed for developing treatments for diseases in humans. The inventors are not aware of any studies that implement stem cell strategies for increasing vigor in domesticated animals.

As provided by the methods of the invention herein, the cells are administered by injecting one or a plurality of stem cells with a syringe, inserting the stem cells with a catheter or surgically implanting the stem cells. In certain embodiments, the stem cells are administered into a body cavity fluidly connected to a target tissue. In certain preferred embodiments, the body cavity is a brain ventricle. In other embodiments, the stem cells are inserted using a syringe or catheter, or surgically implanted directly at the target tissue site. In other embodiments, the stem cells are administered systemically (e.g., parenterally).

A number of different stem cells are appropriate for increasing vigor as taught herein. Examples, include, but are not limited to, mesenchymal stem cells (MeSCs), neural stem cells (NSCs), hematopoietic stem cells (HSCs). U.S. application Ser. Nos. 11/258,401; 11/258,603; 11/258,392 and 11/258,360 discuss various methods for biasing potency and/or differentiation of stem cells, and are incorporated herein by reference. Stem cells may be purchased from commercially available sources, see Neuroreport, Vol 12 No 6 8 May 2001 and Restorative Neurology and Neuroscience 22 (2004) 459-468, or procured from autogenic, allogenic or xenogenic sources according to known techniques, see for example U.S. Patent Publication 20060078993; Br. J. Haematol. (2000) 109, 235-242; and International Patent Application WO 03/070922. Population of stem cells can be derived from multiple sources, including but not limited to, brain-derived neural stem cells, bone marrow derived mesenchymal stem cells, adipose-derived mesenchymal stem cells, blood-derived hematopoietic stem cells, cord-blood-derived stem cells. Cells could be derived from embryonic stem cells following treatment to induce differentiation toward a specific cell lineage.

In view of the teachings herein, one skilled in the art will appreciate that cells may be administered to an animal by a number of methods, including, but not limited direct injection into a target tissue or distal injection from which cells are transported or migrate to a target tissue. In one embodiment, intraventrical injection is performed to increase cognitive function in the brain of a domesticated animal. Neural stem cells are the ideal candidate for intraventrical injection. In an alternative embodiment, mesenchymal stem cells are co-implanted with neural stem cells into the brain, such as by intraventrical injection. The inventors believe that the mesenchymal stem cells implanted before or concurrent to neural stems cells will lessen inflammation and optimize the environment so as to encourage desired migration and/or differentiation of the implanted neural stem cells.

In another embodiment, cells are administered through parenteral injection. In an optimum embodiment, hematopoietic stem cells are injected into the blood. The inventors believe that the hematopoietic stem cells will travel into the blood and broadly revitalize tissues in the subject's body. In particular, the inventors believe that stamina and energy would be increased via this embodiment.

Many domesticated animals are suitable subjects for the techniques described herein, including, but not limited to, dogs, cats, pigs, horses, and cattle. It is contemplated that the animals most suitable as subjects are dogs, cats, and horses. Increasing the vigor of household pets such as older dogs and cats would provide a great benefit and improved life for the pet and pet owners. As for horses, whether used as work animals or competitive racing, certain embodiments of the invention would result in increased stamina and better outcomes for the horses.

In one embodiment where an autologous cell sample is produced, hematopoietic stem cells are procured from the bone marrow of a horse. These procured cells are cultured and expanded to produce an expanded sample of cells. The expanded sample is administered to the bloodstream, muscle tissue, connective tissue, and/or organ of the horse from which they were procured. In an alternative embodiment, an allogenic (same species different subject) cell sample is produced whereby cells from one horse are procured and processed and then administered to a different horse.

In another embodiment, xenogenic cell samples are procured and administered to a pet. In this embodiment, a pet owner having a strong bond with its pet can provide the material to enhance the pet's life, thereby providing a rewarding sense of stewardship in pet owner. In a specific example, mesenchymal stem cells are procured from bone marrow in a pet owner. The stem cells are manipulated to produce a suitable sample for administration. By way of example, cell samples can be administered into the blood of a pet, or the potency of the cells can be increased and then such potent cells can be manipulated to become neural stem cells appropriate for administration to the brain of the pet.

In a specific example, adult somatic cells including but not limited to white blood cells, fibroblasts, mesenchymal stem cells, and skin cells can be treated with nucleotide derivatives such as BrdU or 5-azacytidine to epigenetically modify the cells to increase their developmental potential. Additionally, cells can be treated with genes that expand the potency of cells including but not limited to genes that are responsible for maintaining the properties of embryonic stem cells such as nanog.

Cells can be positively selected for using cell-specific markers including but not limited to CD34, CD133 (hematopoietic stem cells), STRO-1, SH2, SH3, (mesenchymal stem cells), nestin, PSA-NCAM (neural stem cells). Cells can also be purified through negative selection by selecting out cells that express markers not present in the desired cell population. For example, lineage markers indicating differentiation such as CD38, CD45, and “Lin” markers (blood cell lineage proteins expressed in differentiating blood cells) can select out white blood cells from a mixture of cells.

Cells can also be selected using physical properties such as growth characteristics, adhesion, and/or density. For example, a density gradient can separate red blood cells from a solution of bone marrow and adhesion of cells to a culture flask can select for mesenchymal cells (while hematopoietic cells remain non-adherent).

As discussed above, stem cells may be procured using convention techniques in the stem cell art. In one example, stem cells are obtained from bone marrow or blood. See, for example, Friedenstein A J, Gorskaja J F, Kulagina N N, Exp Hematol. September 1976; 4(5):267-74; and Caplan A I J, Orthop Res. September 1991; 9(5):641-50. A bone marrow sample is explanted from a donor and hematopoietic stem cells are isolated from the marrow sample according to known techniques, including use flow cytometry or an affinity column. See, for example, U.S. Patent Publication Nos. 20040265996; 20050158857; 20060088890; and 20060073124. In a specific embodiment, hematopoietic cells are isolated using positive or negative selection. See U.S. Patent Publication No. 20060073124. Negative selection removes unwanted cells using certain markers such as C45 or positive selection using CD34.

Once cells are isolated they may be cultured, expanded, subjected to external biasing factors and/or genetically modified by introduction of genes encoding for biasing factors, see U.S. application Ser. Nos. 11/258,401; 11/258,603; 11/258,392 and 11/258,360 are incorporated herein by reference. Mesenchymal cells may be isolated by similar techniques or through the use of a gradient, such as FICOL gradient. Mesenchymal cells in a bone marrow sample will attach to surface, whereas other undesired cells will not attach and remain in the media. The media with the undesired cells is removed leaving the desired mesenchymal cells. The mesenchymal cells, as with other cells, are cultured, expanded, subjected to external biasing factors and/or genetically modified.

As noted above, cells can be harvested from an animal and autologously transplanted back following treatment and/or expansion of cells. However, it may be more efficient to obtain stem cells from readily accessible source. For example, according to another embodiment, cells are harvested, catalogued according to predetermined characteristics, e.g., genotyping, blood type, major histocompatibility complex, or genomic characteristics, and stored under appropriate conditions (typically by freezing) to keep the stem cells alive and functioning. Essentially, this embodiment pertains to the production of a stem cell bank. The stem cell bank facilitates the selection from a plurality of samples of a specific stem cell sample which provides an optimal match. Thus, another embodiment of the subject invention pertains to a stem cell bank comprising a plurality of stem cells samples obtained from separate sources and which are characterized and catalogued according to at least one predetermined characteristic. An additional embodiment pertains to a method of establishing a stem cell bank comprising collecting stem samples from multiple sources; cataloguing the samples according to at least one predetermined characteristic and storing the cells under conditions that keep cells viable.

The inventors have found that co-administration of secondary cells with primary cells can influencing the transplant loci so as to be more conducible to acceptance and differentiation of the primary cells to their target cell type. For example, mesenchymal cells may reduce the amount of inflammation at the site of implantation of hematopoietic or neural stem cells.

As used herein, the term ‘vigor’ refers to physical strength, stamina, mental strength, robustness of tissue and/or energy in a nonhuman vertebrate. It is contemplated that the increase in vigor is not targeted to a pathology or disease. Accordingly, the methods taught herein can serve as a vigor boost to even a clinically healthy animal.

As used herein, the term ‘reproductive capacity’ refers to fertility and/or successful gestation. For example, increased reproductive capacity for males may refer to increased libido and/or sperm count. Increased reproductive capacity in females may refer to increased likelihood of impregnation and increased birthrate.

As used herein, the term ‘agricultural output’ refers to the amount of animals produced per a given period of time and/or amount of land for a population of animals, or the amount of biomass obtained from a population of animals over a period of time and/or amount of land. Biomass includes but is not limited to flesh or replenishable resources such as wool or hair. In one embodiment, the invention pertains to a method of increasing the reproductive capacity of a cow, horse, pig, sheep, buffalo, or goat comprising administering a vigor-enhancing composition to said cow, horse, pig, sheep, buffalo or goat, said vigor-enhancing composition comprising autologous stem cells or exogenous stem cells, or both, and a pharmaceutically acceptable carrier, and wherein said administering is not targeted to treating a pathology in said animal. In another embodiment, the invention pertains to a method of increasing the agricultural output of a population of non-human animals comprising administering a vigor-enhancing composition to said population, said vigor-enhancing composition comprising autologous stem cells or exogenous stem cells, or both and a pharmaceutically acceptable carrier.

In a further embodiment, stem cells are co-administered with a nutrient composition. The nutrient composition may be administered prior to, concurrent to, or subsequent to the administration of the stem cells. The nutrient composition may be administered in a similar mode as the stem cells or by a separate mode. For example, stem cells may be administered by parenteral injection while the nutrient composition is administered by oral ingestion.

In another embodiment, external factors are co-administered with the stem cells. Typically, such factors are provided at the site of administration. Such external factors may optimize the implantation site environment, or may serve to bias differentiation of the implanted stem cells.

In a further embodiment, the subject invention pertains to a method of increasing the longevity of a healthy nonhuman vertebrate comprising administering a longevity enhancing composition to said nonhuman vertebrate, said longevity-enhancing composition comprising autologous stem cells or exogenous stem cells, or both, and a pharmaceutically acceptable carrier, and wherein said administering is not targeted to treating a pathology in said vertebrate.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

The teachings of all references cited herein are incorporated by reference in their entirety to the extent not inconsistent with the teachings herein.

What is claimed is:

1. A method of increasing the vigor of a healthy nonhuman vertebrate comprising administering a vigor-enhancing composition to said nonhuman vertebrate, said vigor-enhancing composition comprising autologous stem cells or exogenous stem cells, or both, and a pharmaceutically acceptable carrier, and wherein said administering is not targeted to treating a pathology in said vertebrate.

2. The method of claim 1, further comprising extracting a population of stem cells from said mammal to obtain extracted stem cells and culturing said extracted stem cells.

3. The method of claim 1, wherein said stem cells are administered to an equine animal such that stamina or speed, or both, of said equine animal is increased.

4. The method of claim 1, wherein said stem cells are administered parenterally.

5. The method of claim 4, wherein said stem cells are administered intravenously.

6. The method of claim 4, wherein said stem cells are administered intramuscularly.

7. The method of claim 4, wherein said stem cells are administered to muscle, liver, kidney, blood, joint, or brain of said vertebrate.

8. A method of increasing the vigor of a cat or dog comprising administering a vigor-enhancing composition to said cat or dog, said vigor-enhancing composition comprising autologous stem cells or exogenous stem cells, or both, and a pharmaceutically acceptable carrier, and wherein said administering is not targeted to treating a pathology in said animal.

9. A method of increasing cognitive function of a human or non-human animal comprising administering a stem cell composition to brain tissue of said human or non-human animal, said stem cell composition comprising autologous stem cells or exogenous stem cells, or both and a pharmaceutically acceptable carrier, and wherein said administering is not targeted to treating a pathology in said human or non-human animal.

10. The method of claim 9, wherein said administering comprises injection into a ventrical in the brain of said human or non-human animal.

11. The method of claim 1, wherein said nonhuman vertebrate is a dog.

12. The method of claim 1, wherein said nonhuman vertebrate is a cat.

13. The method of claim 1, wherein said nonhuman vertebrate is a ruminant.

14. The method of said claim 13, wherein said ruminant is a cow.

15. The method of claim 13, wherein said ruminant is a goat or sheep.

16. The method of claim 1, wherein said nonhuman vertebrate is a horse.
